(19) United States
(12) Patent Application Publication
(10) Pub. No.: US 2016/0113569 A1
(43) Pub. Date: Apr. 28, 2016

(54) HEALTH ISSUE DETECTION AND TREATMENT SYSTEM

(71) Applicant: EBAY INC., San Jose, CA (US)

(72) Inventors: Lucy Ma Zhao, Austin, TX (US); Kamal Zamer, San Jose, CA (US)

(21) Appl. No.: 14/525,879

(22) Filed: Oct. 28, 2014

(51) Int. Cl. A61B 5/00; G06F 19/00; A61F 5/14; A61N 2/00; A61N 1/36

(52) U.S. Cl. CPC A61B 5/4836; A61B 5/6802; A61N 2/00; A61N 1/36; A61B 5/6898; A61F 5/14; A61B 5/486; A61B 5/7246; A61B 5/7282; G06F 19/3406; G06F 19/345; A61B 2560/0475

(57) ABSTRACT

Systems and methods for health issue detection and treatment include collecting, from at least one wearable user device associated with a first user, user health data at a plurality of different times. Previous user health data retrieved from the at least one wearable user device associated with the first user at a plurality of previous times is analyzed to create a first user health profile that is stored in a database. The current user health data retrieved from the at least one wearable user device associated with the first user at a current time is compared with the first user health profile stored in the database and, in response, a first user health deviation is detected. A first user health treatment is then provided for display on a first user device that is associated with the first user.

### BACKGROUND

1. Field of the Disclosure

The present disclosure generally relates to the treatment of health issues and more particularly to a health issue detection and treatment system.

2. Related Art

Conventional health issue treatment typically involves a user experiencing negative health issues and visiting a doctor once those negative health issues reach a level that the user can no longer ignore. If those negative health issues are serious enough, the user may be monitored over the course of hours, days, or even weeks to attempt to determine the cause of the negative health issues. Such monitoring may be accomplished by having the user hospitalized so that specialized monitoring equipment may be used to collect health data for diagnosis, or by issuing the user portable specialized monitoring equipment that the user may use at home to collect health data for diagnosis. However, such conventional methods require a user to experience negative health issues at a level that convinces the user to visit a doctor, and then monitoring must occur via the specialized monitoring equipment discussed above and the associated health data provided to the doctor so that the user's condition may diagnosed. As such, the diagnosis and subsequent treatment of negative health issues of a user typically does not provide preventative treatment, as by the time a user visits the doctor and begins utilizing the specialized monitoring equipment, the user's condition has progressed to a point where preventative treatment is no longer an option.

Thus, there is a need for an improved health issue detection and treatment system.

### BRIEF DESCRIPTION OF THE FIGURES

FIG. 1 is a flow chart illustrating an embodiment of a method for heath issue detection and treatment;

FIG. 2 is a front view illustrating an embodiment of a user wearing a plurality of wearable user devices;

FIG. 3a is a chart illustrating an embodiment of user health data;

FIG. 3b is a chart illustrating an embodiment of a user health profile created using a plurality of different types of user health data;

FIG. 4a is a chart illustrating an embodiment of current user health data that deviates from a user health profile created using the user health data of FIG. 3a;

FIG. 4b is a chart illustrating an embodiment of current user health data that deviates from the user health profile of FIG. 3b;

FIG. 5 is a screen shot illustrating an embodiment of a user heath treatment screen;

FIG. 6 is a screen shot illustrating an embodiment of a user heath treatment screen;

FIG. 7a is a screen shot illustrating an embodiment of a user heath treatment screen;

FIG. 7b is a screen shot illustrating an embodiment of a merchant finder screen retrieved using the user heath treatment screen of FIG. 7a;

FIG. 8 is a screen shot illustrating an embodiment of a user heath treatment screen;

FIG. 9 is a schematic view illustrating an embodiment of a networked system;

FIG. 10 is a perspective view illustrating an embodiment of a user device;

FIG. 11 is a perspective view illustrating an embodiment of a wearable user device;

FIG. 12 is a schematic view illustrating an embodiment of a computer system; and

FIG. 13 is a schematic view illustrating an embodiment of a system provider device.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

The present disclosure provides systems and methods for detecting health issues and providing health treatments to address those health issues. Those systems and methods utilize wearable user devices, which are regularly worn by a user for functionality other than health issue detection and treatment, in order to generate and store user health data at different times. For any particular time, the user health data previously retrieved may be analyzed to generate a user health profile for the user that is indicative of a healthy user, and the current user health data retrieved may be compared to that user health profile to determine whether a user health deviation is occurring that is indicative of a negative health issue for the user. If a user health deviation is occurring, the systems and methods may provide a user health treatment for display on a user device of the user. The user health treatment may include a variety of different preventative user health treatments, including the suggestion to the user of a medication to remedy the negative health issue, the suggestion to the user of a purchase that has remedied similar user health deviations of other users with similar health profiles, the option for the user to activate a user physical treatment system that may include a magnetic treatment system, an electrical current treatment system, or a user support device physical adjustment system, and/or the sending of a request to a friend of user to meet up with the user. The systems and methods of the present disclosure provide for the early detection of possible negative health issues of a user without the need to visit a doctor and/or obtain specialized monitoring equipment, along with the suggestion of preventative health treatments that may prevent those negative health issues from getting worse.

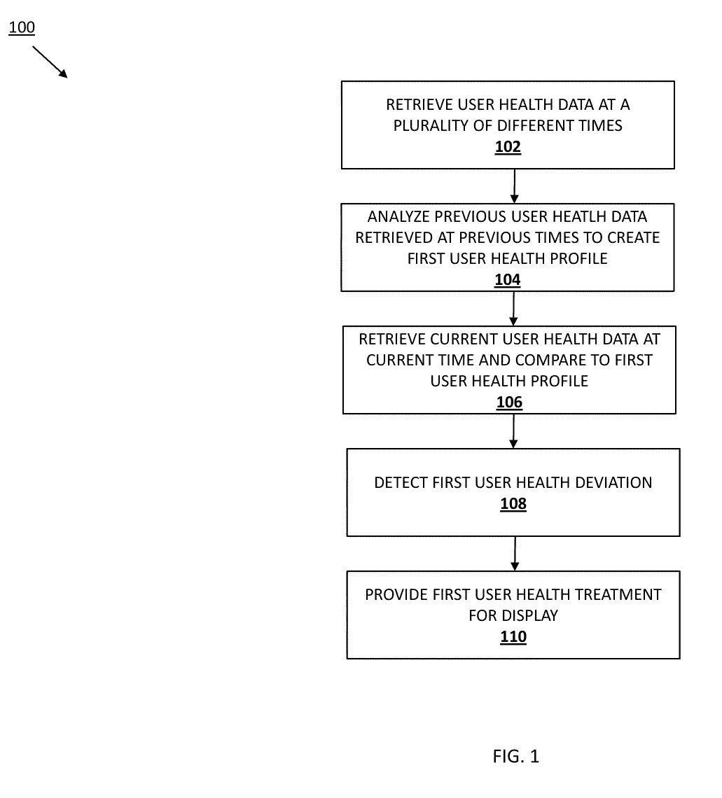

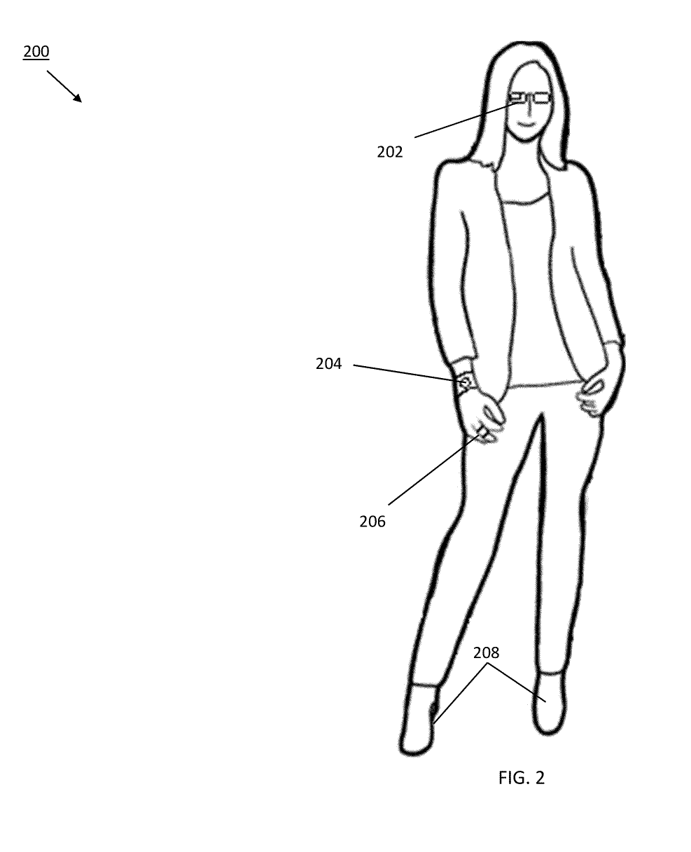

Referring now to FIGS. 1 and 2, an embodiment of a method 100 for health issue detection and treatment is illustrated. In some of the embodiments discussed below, the method 100 is performed by a system provider that is a payment service provider such as, for example, PayPal Inc. of San Jose, Calif., that operates a system provider device that is a payment service provider device. For example, the payment service provider may provider payment services for users and merchant to allow users to make purchases from merchants by transferring funds from user payment accounts (e.g., provided by account providers) to merchant payment accounts (e.g., provided by account providers), and the payment service provider may provide the health issue detection and treatment system as part of those payment services. However, other system providers (e.g., the account providers, health providers, other third party providers, etc.) may provide the health issue detection and treatment systems as part of other services and/or as a stand-along service while remaining within the scope of the present disclosure.

In some embodiments, the user devices (e.g., user mobile phones) discussed below may include a health issue detection and treatment application and/or payment application that operates to cause those user devices to perform at least some of the functions discussed below for participating in the heath issue detection and treatment system. As such, any actions associated with the heath issue detection and treatment system including associating users with wearable user devices, retrieving user health data from the user wearable devices, analyzing previous user health data collected at previous times to create a first user health profile, comparing current health data collected at a current time to the first user health profile, detecting a first user health deviation, providing a first user health treatment for display, determining a connection between users, checking user calendars, activating user physical treatment systems, and/or other actions discussed below, may be performed by or in conjunction with the operation of the heath issue detection and treatment application on the user device. However, in some embodiments, at least some of those actions may be performed by the system provider device and the results of such actions communicated to the user device over a network.

FIG. 2 illustrates a user 200 that, in the embodiments discussed below, is the first user 200 that experiences a negative health issue during the method 200 discussed below. However, one of skill in the art in possession of the present disclosure will recognize that the health issue detection and treatment system may include any plurality of users that utilize the wearable user devices and health issue detection and treatment system substantially as discussed for the first user 200 below. As also discussed below, the user health data collected from multiple users in the health issue detection and treatment system may be stored and analyzed to allow for the determination of user health treatments for users with similar user health profiles and user health deviations, as well as other benefits that would be apparent to one of skill in the art in possession of the present disclosure. The first user 200 includes or has at least one of a plurality of wearable user devices including, in the illustrated embodiment, “smart” glasses 202 such as, for example, Google Glass® available from Google Inc. of Mountain View, Calif., a “smart” watch 204 such as, for example, the Apple Watch available from Apple, Inc. of Cupertino, Calif., a “smart” ring 206 such as, for example, the MOTA smart ring available from MOTA of Sunnyvale, Calif., and “smart” shoes such as, for example, Lechal footwear available from Ducere Technologies Pvt. Ltd. Of Andhra Pradesh, India. While a few examples of wearable user devices are illustrated in FIG. 2 and discussed below, one of skill in the art will recognize that a wide variety of wearable user devices may be embedded into wearable articles (including pants, shirts, jackets, hats, scarfs, jewelry, and/or other wearable articles of the first user 200), and thus those wearable user devices will fall within the scope of the present disclosure.

As would be understood by one of skill in the art, the “smart” wearable user devices may include processing systems, memory systems, communication systems, sensor systems, and/or any other devices or systems known in the art that allow those wearable user devices to collect the user health data and communicate it as discussed below. For example, the “smart” glasses 202 may collect audio data, video data (i.e., from the point of view of the first user 200, of the first user 200 via an eye or face facing camera, etc.), user movement (e.g., head movement) data, brainwave data, temperature data, breathing data, and/or any other user data known in the art that is collectable by “smart” glasses. Similarly, the “smart” watch 204 may collect user movement (e.g., arm and hand movement) data, pulse data, temperature data, and/or any other user data known in the art that is collectable by “smart” watches, the “smart” ring 206 may collect user movement (e.g., arm, hand, and finger movement) data, pulse data, temperature data, and/or any other user data known in the art that is collectable by “smart” rings, and the “smart” shoes 206 may collect user movement (e.g., foot and leg movement such as walking/running movements) data, pulse data, temperature data, and/or any other user data known in the art that is collectable by “smart” shoes.

In addition, the wearable user devices may provide, or the first user 200 may include or have, separate, user physical treatment systems. In some embodiments, the user physical treatment systems may include magnetic treatment systems that provide for the activation of a magnetic force adjacent a portion of the user's body in order to treat that portion of the user's body with the magnetic force. For example, the “smart” shoes 208 may include wire coils and a power source that may be activated to produce a current through the wire coils that generates a magnetic field, and those wire coils may be oriented in the “smart” shoes 208 to direct the magnetic field towards portions of the feet of the first user 200 that may be treated with a magnetic force when the first user 200 is wearing the “smart” shoes 208. Similarly the first user 200 may wear a wrist band, arm band, leg band, chest strap, or other similar user physical treatment devices that include such wire coils and power sources to allow a magnetic field to be produced and directed toward any portion of the body of the first user 200 that responds to therapeutic magnetism treatments. While a few examples of magnetic user physical treatment systems have been described (both as part of the “smart” wearable user devices and separate devices), other methods for generating magnetic forces for treating a user will fall within the scope of the present disclosure.

In some embodiments, the user physical treatment systems may include electric current treatment systems that provide for the activation and application of an electric current to a portion of the user's body in order to treat that portion of the user's body with the electric current. For example, the “smart” glasses 202 may include electrodes and a power source that may be activated to produce an electric current through the electrodes, and those electrodes may be oriented on the head of the first user 200 to direct the electric current towards portions of the brain of the first user 200 that may be treated with an electric current when the first user 200 is wearing the “smart” glasses 202 (e.g., via a non-invasive, painless brain stimulation treatment such as transcranial direct current stimulation (tDCS) that may either excite neuronal activity (anodal stimulation) or inhibit/reduce neuronal activity (cathodal stimulation)). Similarly the first user 200 may wear a wrist band, arm band, leg band, chest strap, or other similar user physical treatment devices that include such electrodes and power sources to allow an electric current to be produced and directed toward any portion of the body of the first user 200 that responds to therapeutic electric current treatments. While a few examples of electric current user physical treatment systems have been described (both as part of the “smart” wearable user devices and separate devices), other methods for generating electric currents and providing them to a user for treating the user will fall within the scope of the present disclosure.

In some embodiments, the user physical treatment systems may include user support device physical adjustment systems that provide for the activation and adjustment of a user support device to a portion of the user's body. For example, the “smart” shoes 208 may include adjustable inserts or other foot engagement or support devices that may be activated to adjust how the “smart” shoes 208 support and/or engage the user's feet. Similarly the first user 200 may wear adjustable compression straps, adjustable back support devices, or other similar user physical treatment devices that include adjustable support systems for any portion of the body of the first user 200 that responds to adjustable support or engagement. While a few examples of user support device physical adjustment systems have been described (both as part of the “smart” wearable user devices and separate devices), other methods for adjusting support or engagement of a user's body for treating the user will fall within the scope of the present disclosure.

Thus, the first user 200 may wear a wide variety of wearable user devices from which a wide variety of user data may be collected, as well as user physical treatment systems that may be activated to treat the first user 200, only some of which are discussed below. As such, while several examples of wearable user devices, user physical treatment systems, collected user data, and user physical treatments are provided herein, one of skill in the art in possession of the present disclosure will recognize that other wearable user devices and user physical treatment systems may be utilized to collect other types of user data that may be used to recommend and/or activate other user physical treatments that will fall within the scope of the present disclosure. In an embodiment, each of the wearable user devices may communicate any user data collected to a user device (e.g., a mobile phone via Bluetooth or other wireless communications techniques) or may transmit that user data directly to a system provider device over a network.

The method 100 begins at block 102 where user health data is retrieved at a plurality of different times. As discussed above with reference to FIG. 2, the first user 200 includes a plurality of wearable user devices that may collect a variety of user health data, and that user health data may be communicated to a storage module or health data collection circuit (which may be configured by a processing system using instructions in a non-transitory computer-readable medium) in the user device of the first user 200 (e.g., a mobile phone, a tablet device, a laptop computer, etc.) and/or a system provider device of the health issue detection and treatment system provider. In some embodiments, user health data may be collected by the “smart” glasses 202 and may include audio data (e.g., recordings of the voice of the first user, recordings of the surrounding of the first user, etc.), video data (e.g., recordings of the point of view of the first user 200, recordings of the eye of the first user 200, recordings of the eye movement or eye reaction of the first user 200, etc.), head movement data, brainwave data, temperature data, breathing data, and/or any other user health data known in the art that may be collectable by “smart” glasses.

In some embodiments, user health data may be collected by the “smart” watch 204 and may include arm and hand movement data, pulse data, temperature data, and/or any other user data known in the art that may be collectable by “smart” watches. In some embodiments, user health data may be collected by the “smart” ring 206 and may include arm, hand, and finger movement data, pulse data, temperature data, and/or any other user data known in the art that may be collectable by “smart” rings. In some embodiments, user health data may be collected by the “smart” shoes 206 and may include foot and leg movement data, pulse data, temperature data, walking/running data, and/or any other user data known in the art that may be collectable by “smart” shoes. Any or all of the user data may be retrieved by the storage module or the health data collection circuit in the user device and/or the system provider device and stored in a storage system, a non-transitory memory, and/or a database.

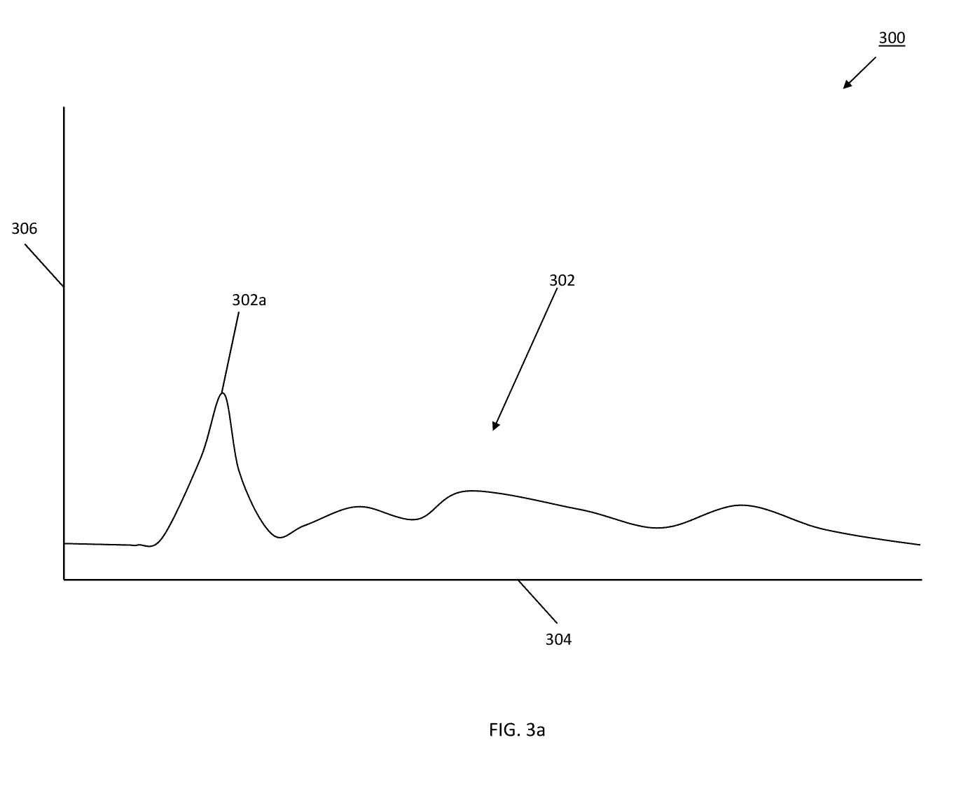

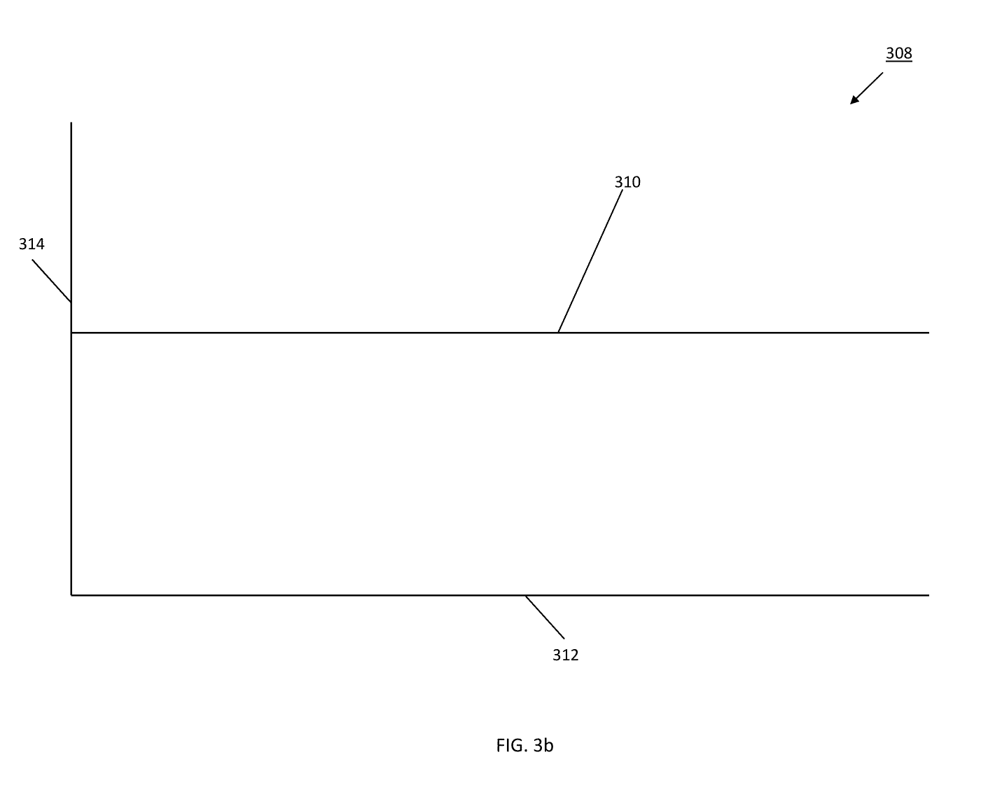

Referring now to FIG. 3a, an embodiment of a chart 300 is illustrated that includes a plurality of user health data 302 that may be provided by a single set of user health data or by multiple sets of user health data. The chart 300 includes an X-axis 304 that, in the illustrated embodiment, measures time, and a Y-axis 306 that may measure a variety of different user health parameters. For example, in some embodiments, the Y-axis 306 may measure a user's heart rate, and the user health data 302 provides the user's heart rate over time (e.g., a day).

In one example of the Y-axis 306 measuring a user's heart rate, the user health data 302 may include a spike 302a that may be indicative of a user exercising (e.g., relatively early in the morning) and/or other heart rate intensive activity by the first user 200. In other examples, the Y-axis 306 may measure a user's breathing rate such that the user health data 302 provides the user's breathing rate over time, a user's brainwave activity such that the user health data 302 provides the user's brainwave activity over time, a user's temperature such that the user health data 302 provides the user's temperature over time, and/or some other physiological user data such that the user health data 302 provides the physiological user data over time for the first user 200.

Furthermore, the chart 300 illustrating and described above as provided using physiological data is just an example, and non-physiological data that may or may not be displayable in a similar chart may be collected at block 102. For example, the audio data of a user's voice or a user's surroundings may be considered user health data, or that audio data of the user's voice or surroundings may be collected and analyzed to determine stress (or other parameters) that may be visualized in a chart similar to the chart 300. In another example, the video data of a user's point of view, eye movement, or eye response may be considered user health data, or that video data of a user's point of view, eye movement, or eye response may be analyzed to determine stress (or other parameters) that may be visualized in a chart similar to the chart 300. In another example, data of a user's movement (e.g., walking data, arm movement data, etc.) may be considered user health data, or that data of a user's movement may be analyzed to determine relative or absolute levels of activity (or other parameters) that may be visualized in a chart similar to the chart 300. Thus, any user data that is detectable by the wearable user devices may be retrieved over time from the wearable user devices and stored by the storage module or health data collection circuit in the user device or the system provider device, and following block 102, a plurality of previous user health data has been retrieved at a plurality of previous times and stored in a database.

Referring now to FIGS. 3a and 3b, the method 100 then proceeds to block 104 where previous user health data retrieved at previous times is analyzed to create a first user health profile. In an embodiment, following the retrieval of user health data from any of the wearable user devices at any given time, a health profile determination module or profile determination circuit (which may be configured by a processing system using instructions in a non-transitory computer-readable medium) in the user device and/or the system provider device may analyze the previous user health data that was retrieved at a plurality of previous times to create a user health profile. A wide variety of user health data may be used by itself or in combination with other user health data to create one or more user health profiles, and each of those user health profiles attempt to quantify what it means for the first user 200 to be “healthy”. As such, some particular sets of user health data may not be used in creating the user health profile (e.g., user health data that is outside a range that includes the majority of the user health data) because those particular sets of user health data may be user health data indicative of an unhealthy first user 200, may be user health data outside the normal range of the majority of user health data, and/or may otherwise be not indicative of the normal health of the user.

In an embodiment, the chart 300 illustrating in FIG. 3 includes collected user health data 302 that provides a user health profile. For example, the health profile determination module or profile determination circuit in the user device and/or system provider device may analyze the collected user health data collected over a plurality of days, and may determine that a majority of that user health data provides a normalized curve indicated by the user health data 302. In a specific example, the user health data 302 in the chart 300 may be collected over a plurality of weeks or months and may be indicative of a user's heart rate on Mondays. As such, a user health profile may be created that includes the normalized curve indicated by the user health data 302, and that that normalized curve may be indicative of the first user typically exercising on Monday mornings. In this specific example, there may be a few sets of user health data in which the first user 200 did not exercise on Monday mornings, and because the majority of user health data was indicative of the first user exercising on Monday mornings, those sets of user health data may have been dropped or otherwise not included in the creation of the user health profile that includes the normalized curve indicated by the user health data 302 in the chart 300.

One of skill in the art will recognize how user health profiles may thus be created for any user health data that is collected such that user health profiles are created and stored that indicate the “healthy”, normal, or average heart rate of the first user 200 for each day of the week, hour of the day, etc., the “healthy”, normal, or average breathing rate of the first user 200 for each day of the week, hour of the day, etc., the “healthy”, normal, or average brainwave activity of the first user 200 for each day of the week, hour of the day, etc., the “healthy”, normal, or average temperature of the first user 200 for each day of the week, hour of the day, etc., the “healthy”, normal, or average movement of the first user 200 for each day of the week, hour of the day, etc., the “healthy”, normal, or average occurrence of laughter by the first user 200 for each day of the week, hour of the day, etc., the “healthy”, normal, or average eye movement and response of the first user 200 for each day of the week, hour of the day, etc. and/or for any other “healthy”, normal, or average individual parameters measured by one or more of the wearable user devices discussed above. Such profiles can also be based on location, event, and/or other factor. For example, a user may have different health profiles on vacation, at work, while sleeping, at a gym, presenting at a conference, driving in rush hour traffic, shopping on Christmas Eve, etc.

Referring now to FIG. 3b, an embodiment of a chart 308 is illustrated and includes a user health profile 310 that is created from a plurality of different types of user health data. For example, the chart 308 includes an X-axis 312 that, in the illustrated embodiment, measures time, and a Y-axis 314 that may provide a normalized, multi-parameter measure of the plurality of user health data. For example, in some embodiments, the Y-axis 306 may provide a normalized multi-parameter measure of a user's “healthy”, normal, or average heart rate, a user's “healthy”, normal, or average breathing rate, a user's “healthy”, normal, or average brainwave activity, a user's “healthy”, normal, or average temperature, and/or other “healthy”, normal, or average physiological or non-physiological user data that has been selected based on its likelihood of providing user parameters that are indicative of a healthy first user 200. As such, each of those user health data sets may have had outlier user health data (user health data outside of a predetermined range of the majority of the user health data) purged before being incorporated into the user health profile 310.

While a few examples have been provided, one of skill in the art in possession of the present disclosure will recognize that any number of user health profiles may be created using the same types of user health data (e.g., heart rate data) or combinations of different types of user health data, and those user health profiles stored in a database. Those user health profiles may also be updated whenever new user health data is retrieved (e.g., the current user health data discussed below) that is indicative of a healthy first user 200. Thus, following block 104, at least one first user health profile that was created using a plurality of previous user health data collected at a plurality of previous times is stored in a database that is accessible by the user device and/or the system provider device.

The method 100 then proceeds to block 106 where current user health data is retrieved at a current time and compared to the first user health profile created at block 104. The current user health data may be retrieved at block 106 at the current time in substantially the same manner as discussed above with regard to block 102, the difference being that, at any particular current time, the current user health data will be collected relative to the previous user health data that was used to create the first user health profile as discussed above with reference to block 104. In an embodiment, the user health data collection module or health data collection circuit in the user device and/or the system provider device retrieves the current user health data and, a health issue detection module or detection circuit (which may be configured by a processing system using instructions in a non-transitory computer-readable medium) in the user device or system provider device compares it to the first user health profile in the database. When a user has multiple health profiles, an appropriate health profile for the user is determined, e.g., if the user is currently on vacation, an appropriate health profile may be a profile based on the user being on vacation or a specific type of vacation that corresponds to the current vacation. The comparison of the current user health data with the first user health profile is illustrated and described below for the detection of a first user health deviation. However, in many cases, the current user health data may be retrieved and compared to the first user health profile and found to be substantially similar (e.g., within a predetermined range of the user health data in the first user health profile). In such cases, the current user health data may be added to the first user health profile in response to determining that it is within the predetermined range of the other user health data in the first user health profile or otherwise indicative of a healthy first user 200.

Thus, current user health data may be retrieved on demand, at predetermined intervals (e.g., daily, hourly, every minute, etc.), and/or continuously and, in response, compared to the most recent first user health profile(s) that are stored in the database and that were created using a plurality of previous user health data. Substantial similarity between the current user health data and the first user health profile(s) may be determined in a variety of manners, including defining a predetermined “normal” range using a majority of the previous user health data, using user health profiles collected from other users that are similar to the first user (e.g., that are close in age, height, weight, activity level, etc.), and/or using a variety of other methods known in the art. As such, one of skill in the art in possession of the present disclosure will recognize that current user health data may be determined to be substantially similar to the first user health profile in a variety of manners known in the art such that it is concluded to be indicative of a healthy first user 200.

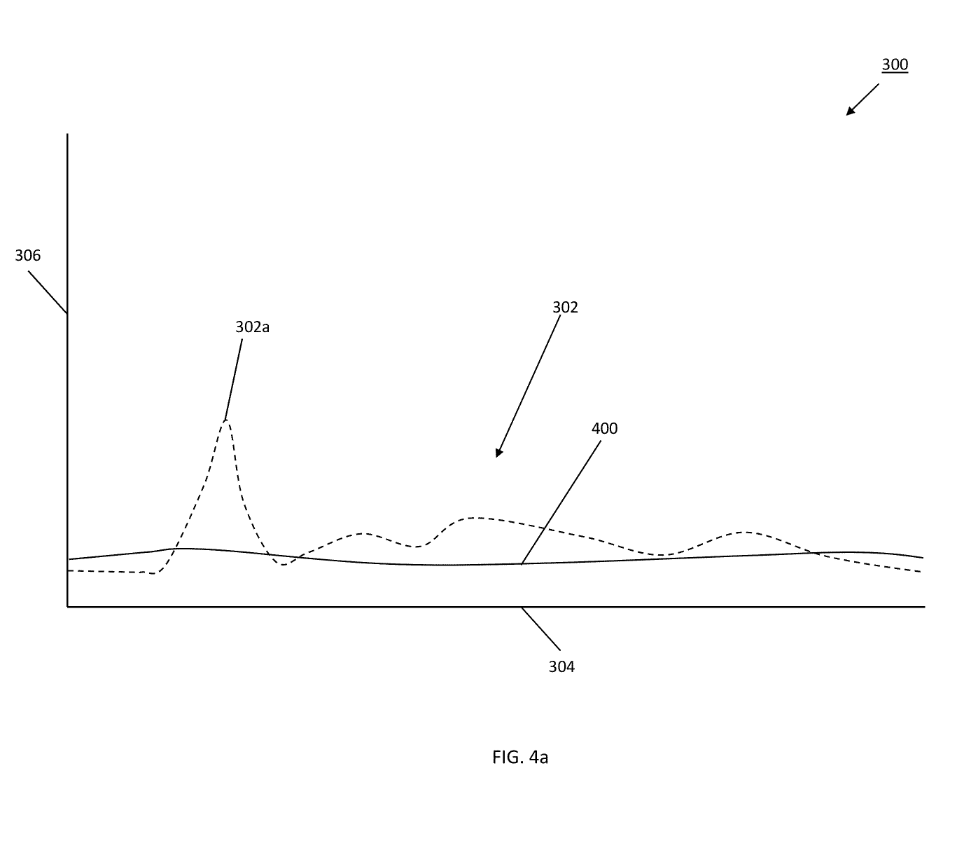

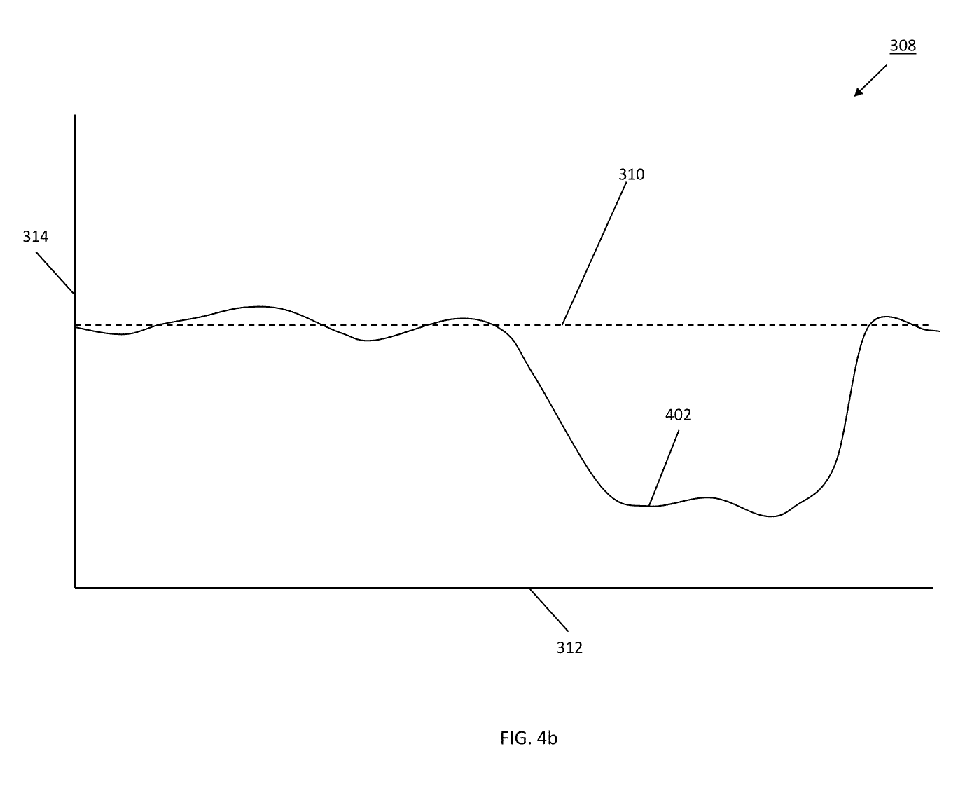

Referring now to FIGS. 4a and 4b, the method 100 then proceeds to block 108 where a first user health deviation is detected. Similarly as discussed above, at block 108 the health issue detection module or detection circuit in user device and/or the system provider device compares the current user health data to the first user health profile in the database. FIG. 4a illustrates a first user health profile that includes a curve indicated by the previous user health data 302 (indicated by the dotted line) that was retrieved at previous times and analyzed to provide the first user health profile, along with current user health data 400 (indicated by the solid line) that was retrieved at a current time. As can be seen, the current user health data 400 differs from the first user health profile provided by the previous user health data (e.g., the spike 302a is not present in the current user health data 400), and such differences may be detected by the health issue detection module or detection circuit in the user device and/or the system provider device as a first user health deviation. Using the example provided above where the Y-axis 306 measures a user's heart rate and the X-axis measures time throughout the day on Mondays, the current user health data 400 may be indicative that the first user 200 has skipped exercising on a Monday. However, the comparison of similar current user health data and first user health profiles may be indicative of an irregular heartbeat for the first user 200, an irregular breathing pattern for the first user 200, an irregular brainwave pattern for the first user 200 (e.g., due to the first user 200 not sleeping), an irregular temperature for the first user 200, irregular movement for the for the first user 200, etc.

FIG. 4b illustrates the first user health profile 310 (indicated by the dotted line) that was created from a plurality of different types of previous user health data that were retrieved at previous times and normalized, along with matching types (relative to the previous user health data used to create the first user health profile 310) of current user health data 402 (indicated by the solid line) that were retrieved at a current time and normalized in the same manner as the previous user health data that was used to create the first user health profile 310. As can be seen, the current user health data 402 differs from the first user health profile 310 (e.g., there is a dip in the normalized value of the current user health data 402 later in the day), and such differences may be detected by the health issue detection module or detection circuit in the user device and/or the system provider device as a first user health deviation. User health profiles created using a plurality of different sets and/or different types of user health data may be utilized to determine user health deviations that may be considered more accurate due to the need for several user health parameters to be outside of their normal range in order for a user health deviation to be detected. For example, a breathing rate deviation, by itself, may not be sufficient to cause the curve created using the current health data 402 to deviate sufficiently from the first user health profile 310 in FIG. 4b to detect a user health deviation, but a breathing rate deviation accompanied by a temperature deviation and a user movement deviation may deviate substantially from the first user health profile 310 and be indicative of the onset of a negative user health issue that may be detected at block 108.

Thus, at blocks 106 and 108, the user device and/or system provider device may operate via various modules and/or configured circuits to retrieve current user health data, compare that user health data to the first user health profile created at block 104, and detect a first user health deviation. User health deviations between the current user health data and the first user health profile(s) may be determined in a variety of manners, including defining a predetermined “normal” range using a majority of the previous user health data, using user health profiles retrieved from other users that are similar to the first user (e.g., that are close in age, height, weight, activity level, etc.), and/or using a variety of other methods known in the art. As such, one of skill in the art in possession of the present disclosure will recognize that current user health data may be determined to be substantially different from the first user health profile in a variety of manners known in the art such that it is concluded that a first user health deviation exists.

The method 100 then proceeds to block 110 where a first user health treatment is provided for display. In an embodiment of block 110, a health treatment module or treatment circuit (which may be configured by a processing system using instructions in a non-transitory computer-readable medium) in the user device and/or the system provider device uses the first user heath deviation detected at block 108 to determine a first user health treatment and provide that first user health treatment for display on the user device of the first user 200. A few examples of first user health treatment provided for display on the user device of the first user 200 are provided below, but one of skill in the art in possession of the present disclosure will recognize that, depending on the first user health deviation detected at block 108, a wide variety of different types of user health treatments may be provided for display on the user device of the first user 200 that will be directed toward remedying or treating that first user health deviation.

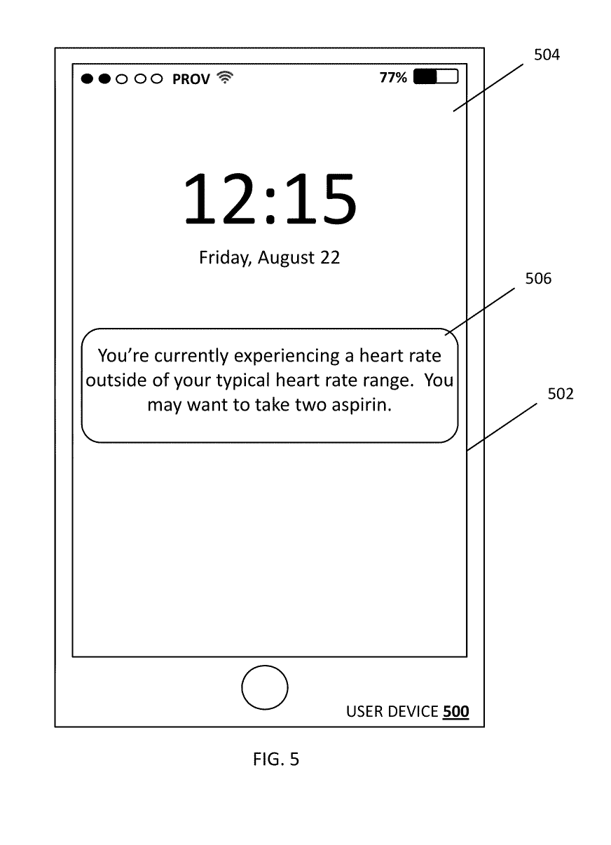

Referring now to FIG. 5, an embodiment of a user device 500 is illustrated that includes a display device 502 displaying a first user health treatment screen 504. As discussed above, the user device 500 may be a mobile phone of the first user 200, but may also be a variety of other user devices known in the art. In the illustrated embodiment, the first user health treatment screen 504 includes a “pop-up” window 506 that is provided on a lock screen (e.g., a user interface element provided by an operating system that regulates immediate access to the user device 500 by requiring that the user perform some action), but may be provided via a user health issue detection and treatment application, as a pop-up on a home screen, and/or using a variety of other display methods known in the art. In the specific example illustrated, the first user health deviation detected at block 108 resulted from current user health data of a heart rate of the first user 200 that deviated from a first user health profile of the “healthy”, “normal”, or average heart rate of the first user 200, and the pop-up window 506 included on the first user health treatment screen 504 informs the first user 200 that their current heart rate is outside of their typical heart rate range, as well as recommending that the first user 200 take two aspirin. In an embodiment, the recommendation to take medication may be determined by the health issue detection module or detection circuit in the user device or system provider device by, for example, referencing a database of user health treatments using the first user health deviation determined at block 108, sending a request including the first user health deviation and receiving a response including the recommendation from a health professional, and/or using a variety of other medication recommendation techniques known in the art.

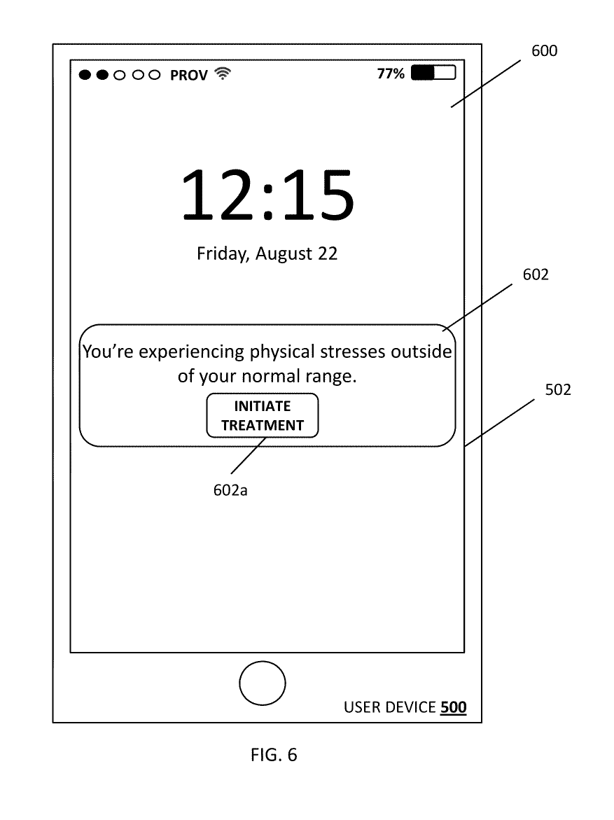

Referring now to FIG. 6, an embodiment of the user device 500 is illustrated displaying a first user health treatment screen 600 on the display device 502. In the illustrated embodiment, the first user health treatment screen 600 includes the “pop-up” window 602 that is provided on the lock screen, but may be provided via a user health issue detection and treatment application, as a pop-up on a home screen, and/or using a variety of other display or notification (including audio) methods known in the art. In the specific example illustrated, the first user health deviation detected at block 108 resulted from current user health data that was indicative of physical stresses of the first user 200 that deviated from a first user health profile of typical physical stresses of the first user 200, and the pop-up window 602 included on the first user health treatment screen 600 informs the first user 200 that they are currently experiencing physical stresses outside of their typical physical stress range. The pop-up window also provides an initiate treatment button 602a that the first user 200 may select to activate a user physical treatment system.

As discussed above, the first user 200 may include any of a variety of user physical treatment systems such as the magnetic treatment system, the electrical current treatment system, and/or the user support device physical adjustment systems discussed above. A physical treatment module or user physical treatment circuit (which may be configured by a processing system using instructions in a non-transitory computer-readable medium) in the user device or system provider device may reference a database of user physical treatments using the first user health deviation detected at block 108 in order to determine one or more user physical treatments that may be initiated by the user physical treatment systems in response to the selection of the initiate treatment button 602.

For example, the modules and/or circuits in the user device and/or system provider device may detect a first user health deviation based on current user health data that includes user movement (e.g., user walking and/or running) that is a deviation from their first user health profile (e.g., the user may have been detected walking several miles further than they do on any average or normal day), and provide the first user health treatment screen 600. In response to the first user 200 selecting the initiate treatment button 602a, the user device 500 may then communicate (e.g., via Bluetooth) with the magnetic treatment system in the “smart” shoes 208 of the first user 200 to activate that magnetic treatment system that is directed to the feet of the first user 200. As such, the magnetic treatment system may be activated in response to the first user health treatment provided at block 110, and may operate to perform a treatment on the user's feet following the detection of a user health deviation that is indicative of sore, aching, or otherwise unhealthy feet of the first user 200. One of skill in the art in possession of the present disclosure will recognize how similar activation of the magnetic treatment system may be utilized in providing magnetic treatments to any other part of the body of the first user 200.

In another example, the user device and/or system provider device may detect a first user health deviation based on current user health data that includes user brainwave activity (e.g., a user headache) that is a deviation from their first user health profile, and provide the first user health treatment screen 600. In response to the first user 200 selecting the initiate treatment button 602a, the user device 500 may then communicate (e.g., via Bluetooth) with the electric current treatment system in the “smart” glasses 202 of the first user 200 to activate that electric current treatment system that is directed to the brain of the first user 200. As such, the electrical current treatment system may be activated in response to the first user health treatment provided at block 110, and may operate to perform a treatment on the user's head following the detection of a user health deviation that is indicative of the first user 200 experiencing a headache. One of skill in the art in possession of the present disclosure will recognize how similar activation of the electrical current treatment system may be utilized in providing electrical current treatments to any other part of the body of the first user 200.

In another example, the user device and/or system provider device may detect a first user health deviation based on current user health data that includes multiple user actions that are indicative of user back pain, and provide the first user health treatment screen 600. For example, current user health data may indicate user movement of the arms and hands that indicate that the user is repeatedly grabbing their back and/or walking awkwardly, audio data of the user mentioning their back, and/or any other data that is indicative of a back issue in the first user 200. In response to the first user 200 selecting the initiate treatment button 602a, the user device 500 may then communicate (e.g., via Bluetooth) with the user support device physical adjustment system in the “smart” shoes 202 of the first user 200 to activate that user support device physical adjustment system to adjust the inserts or supports in the “smart” shoes 202 to alter the support of the feet of the first user 200 in order to reduce the back issues being experienced by the first user 200. As such, the user support device physical adjustment system may be activated in response to the first user health treatment provided at block 110, and may operate to adjust the support or engagement with any part of the body of the first user following the detection of a user health deviation that is indicative of the first user 200 experiencing a back ache.

Furthermore, any combination of the user physical treatment systems included on the first user 200 may be activated in response to the selection of the initiate treatment button 602a on the first user health treatment screen 600. For example, the wearable user devices on the first user 200 may provide user health data that is indicative of a muscle cramp in the user's leg, and each of the magnetic treatment system, the electrical current treatment system, and the user support device physical adjustment system may be activated to, for example, provide a magnetic treatment to the muscle cramp, provide an electrical current treatment to the muscle cramp, and adjust support to the portion of the body of the first user 200 that is having the muscle cramp. One of skill in the art in possession of the present disclosure will recognize how multiple user treatments may be associated with any user health deviation in a database, and retrieved and executed by the user physical treatment systems substantially as discussed above.

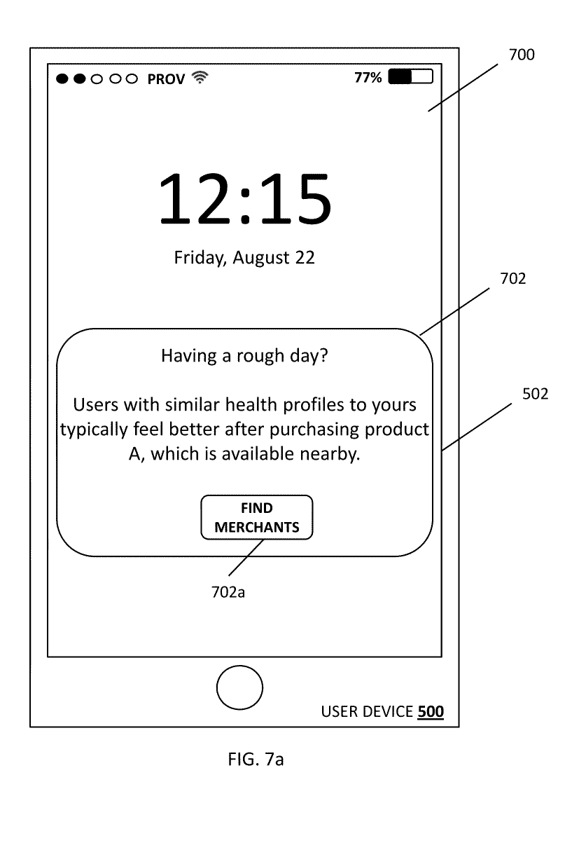

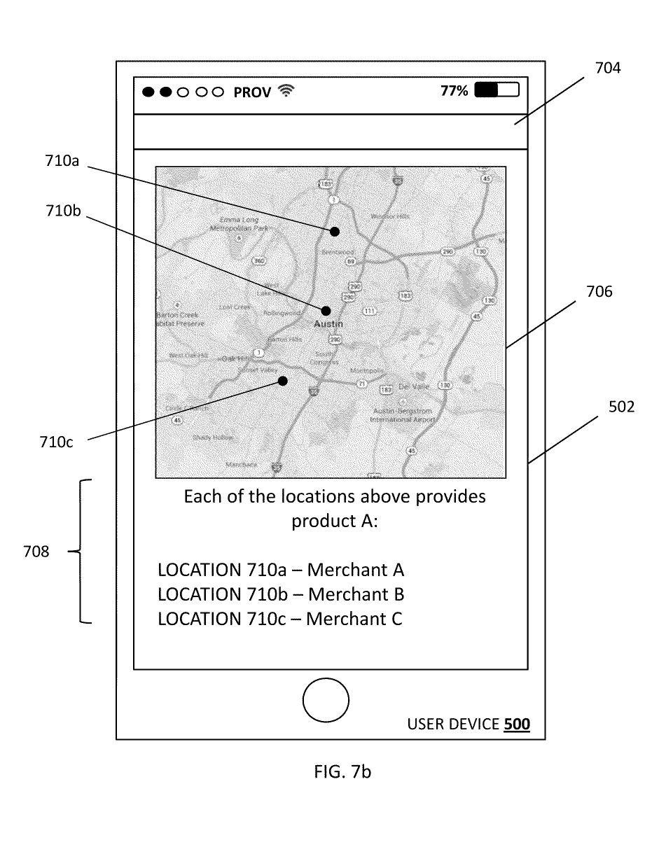

Referring now to FIG. 7a, an embodiment of the user device 500 is illustrated displaying a first user health treatment screen 700 on the display device 502. In the illustrated embodiment, the first user health treatment screen 700 includes the “pop-up” window 702 that is provided on the lock screen, but may be provided via a user health issue detection and treatment application, as a pop-up on a home screen, and/or using a variety of other display methods known in the art. In the specific example illustrated, the first user health deviation detected at block 108 resulted from current user health data that was indicative of a level of happiness of the first user 200 that deviated from a first user health profile of “healthy”, “normal” or average happiness of the first user 200 (e.g., by detecting a lack of laughter, smiling, positive words spoken by the first user 200, movement, etc.), and the pop-up window 702 included on the first user health treatment screen 700 provides a purchase suggestion that informs the first user 200 that similar users have found a particular purchase (“product A” in the illustrated embodiment) helpful in changing their mood, and that purchase is available near the current location of the first user 200. The pop-up window also provides a find merchants button 702a that the first user 200 may select to find merchants at which the particular purchase may be made.

In some embodiments, the system provider device may include a database of user health profiles that have been created for a plurality of different users, as well as user health deviations experienced by those users. In addition, as discussed above, the system provider may be a payment service provider that has access to purchase histories and/or other payment actions of those users. As such, when the first user 200 with the first user health profile experiences the first user health deviation, a purchase treatment module or purchase treatment circuit in the system provider device may reference the database to determine a subset of other users with the same or similar user health profiles and user health deviations, and then reference the purchase histories of that subset of other users to determine purchases made around the time of their respective user health deviations. In the event those purchases are followed by correction, remediation, or other reversal of their user health deviations, the system provider device may determine that those purchases may be provided as a treatment to the first user 200 and provide purchase suggestions for that purchase to the user device 500 for display to the first user 200. As such, purchase suggestions may be provided to the first user that may suggest products and/or services that have proven to remedy similar user health deviations experiences by users with similar user health profiles to the first user 200. For example, purchase suggestions may suggest user purchases of medication for particular user health deviations (e.g., headaches, continued sneezing, etc.), user purchases of massage services for particular user health deviations (e.g., the muscle cramps or back issues discussed above), and even user purchases of clothing (e.g., shoes) for particular user heath deviations (e.g., depression, lethargy, general unhappiness, etc.) if those purchases have been found to reverse similar user health deviations in similar users.

Referring now to FIG. 7b, in response to the user selecting the find merchants button 702a, a merchant finding module or merchant finding circuit in the user device 500 may provide a merchant finder screen 704 that includes a map 706 of a local area in which the user device 500 is located. The merchant finder screen 704 also includes a merchant information section 708 that identifies nearby merchants where the product in the purchase suggestion provided on the first user health treatment screen 700 (e.g., product A in the illustrated embodiment) can be purchased, as well as the relative locations points 710a, 710b, and 710c on the map 706 where those merchants are located. While an embodiment is illustrated of providing physical merchant locations where a purchase can be made, in some embodiments, the merchant finder screen 704 may include links to merchant's websites where the product in the purchase suggestion may be purchased. However, in some embodiments, the therapeutic effects of purchase recommendations may be specific to receiving a physical product or service, and thus the provision of physical merchant locations where the product or service in the purchase suggestion may be purchased may provide particular benefits.

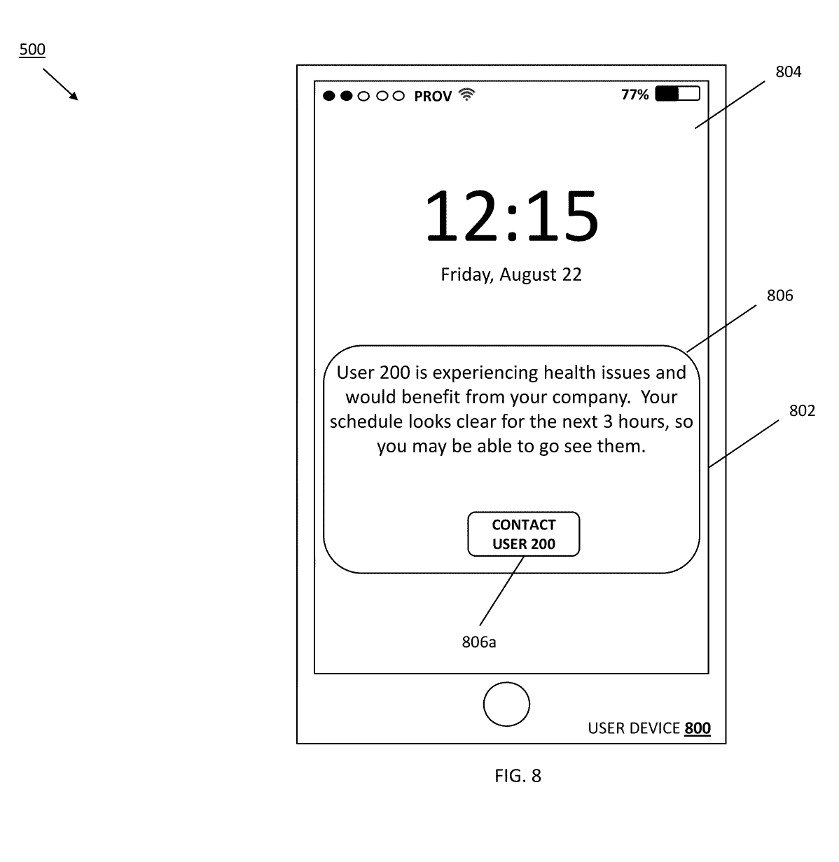

Referring now to FIG. 8, an embodiment of a user device 800 is illustrated that includes a display device 802 displaying a first user health treatment screen 804. The user device 800 may be substantially similar to the user device 500 discussed above, but may belong to a second user that is a friend or otherwise connected to the first user 200. In the illustrated embodiment, the first user health treatment screen 804 includes a “pop-up” window 806 that is provided on a lock screen (e.g., a user interface element provided by an operating system that regulates immediate access to the user device by requiring that the user perform some action), but may be provided via a user health issue detection and treatment application, as a pop-up on a home screen, and/or using a variety of other display/notification methods known in the art. In the specific example illustrated, the first user health deviation was detected at block 108 based on current user health data of the first user 200 that deviated from a first user health profile of the first user 200, and the pop-up window 806 included on the first user health treatment screen 804 is provided on the second user device 800 of a second user that is connected to the first user 200 (e.g., via a communication module or communication circuit in the user device 500 or the system provider device). In the illustrated embodiment, the first user health treatment screen 804 informs the second user that the first user is experiencing health issues and would benefit from the company of the second user. For example, the first user 200 may be detected as being unhappy, depressed, or experiencing some other negative health issue where the company of a friend or relative may be beneficial. The pop-up window 806 included on the first user health treatment screen 804 also includes a contact user button 806a that the second user may select to call, text, email, or otherwise contact the first user 200.

In an embodiment, following the detection of the first user health deviation at block 108, a user connection module or user connection circuit (which may be configured by a processing system using instructions in a non-transitory computer-readable medium) in the user device and/or the system provider device may determine a connection between the first user 200 and at least one second user. For example, the connection between the first user 200 and the second user may be determined using contact information on the user device 500 of the first user 200. In another example, the connection between the first user 200 and the second user may be determined using social network information that may be retrieved using identification and/or authorization information about the first user 200 that is communicated by the user device 500. Social network information used to determine a connection between the first user 500 and second users may include friend designations, common photos or photos including each other, conversations including each other, and/or a variety of other social network information known in the art. In another example, the connection between the first user 200 and the second user may be determined using location information previously retrieved from the user device 500 and user device 800. For example, first user devices of first user and the second user device of the second user may report their locations to the system provider device periodically, which allows the system provider device to store information related to when the second user device is co-located with first user device. That co-location information may then be analyzed by the system provider device to determine which second users are connected with the first user 200 (e.g., which first user devices share a significant common location history with the second user device such that the first user 200 and the second user may be determined to be associated as friends, relatives, etc.)

In some embodiments, the user connection module or user connection circuit in the user device or system provider device may access (e.g., via the Internet) the user device 800 and retrieve a calendar of the second user. The calendar of the second user may then be checked to determine whether the second user is currently available to meet up with the first user 200 (i.e., perform a first user health treatment). As such, the user connection module or user connection circuit in the user device or system provider device may determine a plurality of connections between second users and the first user, and determine which of those second users that are connected to the first user are currently available to meet up with the first user 200. The subset of the second users that are connected to the first user 200 and available to meet up with the first user 200 may be subject to some prioritization or ranking such that higher priority or higher ranked second users are contacted prior to lower priority or lower ranked second users.

While a few examples have been provided, any of a variety of information about and actions by the first user 200 and second users may be analyzed by the system provider device to determine that a connection exists between the first user and the second user. Furthermore, combinations of the information about and actions by the first user 200 and the second users discussed above may be used to determine a degree of confidence that the first user 200 is associated with a second user, and that degree of confidence may be required to reach a predetermined level in order to determine an association between the first user 200 and the second user that is used to provide the first user health treatment screen 804 discussed above. For example, implicit links between users (e.g., being contacts in a mobile phone) may be utilized with other information (e.g., appearance in each other photos on a social network, significant common location histories, etc.) to determine whether the first user health treatment screen 804 should be provided for display to the second user on the user device 800.

Thus, health issue detection and treatment systems and methods have been described that may operate to retrieve user health data from one or more wearable user devices of a user and analyze that user health data to determine a user health profile that is indicative of the user being healthy. Current user health data may then be retrieved and compared to the user health profile to detect when user health deviation of the current user health data from the user health profile. In response to the detection of the user health deviation, a user health treatment is provided for display to the user that may recommend medication to remedy the user health deviation, that may recommend a purchase that is known to remedy the user health deviation, and/or that may provide the ability of the user to initiate a user physical treatment to remedy the user health deviation. In addition, the user's friends may be requested to join the user if doing so may remedy the user health deviation. As such, the health issue detection and treatment systems and methods provide benefits over conventional health issue detection and treatment in that user health deviations that are indicative of negative health issues are detected early, and preventative treatment is recommended immediately, without the need to visit a doctor and/or acquire specialized monitoring equipment.

A few use cases of the health issue detection and treatment system will now be provided, but those use cases should not be interpreted as limiting, as the wide variety of wearable user devices and user physical treatment systems that are and/or may become available for users may greatly expand the type or accuracy of user health data that may be collected and the types of user health treatments that may be performed.

In one use case, the heath issue detection and treatment system collects and monitors purchases and user health data from its user base to understand how purchases by users effect their user health profiles over time, which allows the system to associate user health deviations from user health profiles with purchases of products and/or services that corrected those user health deviations. A particular user may then experience a user health deviation, and that user health deviation may be used to determine one or more purchases of products and/or services that have previously remedied similar user health deviations. Those purchases of products and/or services may then be recommended to that particular user. The purchases of products and/or services may be further refined by only retrieving purchases made by users with similar health profiles or user characteristics (e.g., age, weight, height, activity level, etc.) that experienced similar user health deviations. As such, user health treatments may be “crowd sourced”, which provides for the determination of new remedies to user health deviations for particular users or types of users. In a specific example, a first user may be a female user that is young and relatively active, and experiencing mild depression that is detected by a lower than normal level of activity, a higher than normal level of crying, and a lower than normal level of eating. The system may determine that similar users (young, active females) have recovered from similar cases of mild depression following the purchase of athletic gear such as running shoes or running shorts, and thus the system may provide a user health treatment for display on a user device of the first user that recommends a purchase of running shoes or running shorts.

In another use case, a particular user may occasionally experience chronic pain, and the health issue detection and treatment system may operate to monitor the user to detect when that chronic pain is affecting the user based on lower than normal user movement, modified movement of the user (relative to normal user movement) to compensate for the pain, and complaints from the user. When such chronic pain is detected, the system may provide the user the ability to initiate an electrical current treatment such as tDCS, discussed above, in order to treat the chronic pain. The system may also use multiple detections of the chronic pain in order to try to determine precursor user health data that typically comes before the chronic pain is detected, and once identified, such precursor user health data may be used to suggest and/or provide preventative electrical current treatments prior to or at the very onset of chronic pain in the user.

In another use case, a particular user may have a long and stressful day, and the health issue detection and treatment system may operate to monitor the user to detect higher than normal user movement, higher than normal temperatures of the user, and stress levels from audio collected from the user. When such high stress is detected, the system may provide the user the ability to initiate a magnetic treatment, such as on the user's feet, in order to soothe the user. The system may also use multiple detections of the high stress in the user in order to try to determine precursor user health data that typically leads up to a high stress situations, and once identified, such precursor user health data may be used to suggest and/or provide preventative suggestions to the user to take some time out and relax when they are pushing themselves too hard.

In another use case, the heath issue detection and treatment system collects and monitors user health data from users when they are together to understand how different user company affects their respective user health profiles over time, which allows the system to associate users with other users that make them more relaxed or otherwise healthy (e.g., detected by lower heart rates, laughing, longer deeper breathes, etc.). That user may then experience a user health deviation, and that user health deviation may be used to send a request to one of those other users to join the user in order to regulate their health profile. As such, users may be alerted that they may want to join one of their friends in order to relax the user or otherwise remedy a negative health issue such as depression, anxiety, etc. The system may include prioritization of other users (based on the level of relaxation they provide) for the user and may check the availability of those other users prior to suggesting that they join a user experiencing a user health deviation.

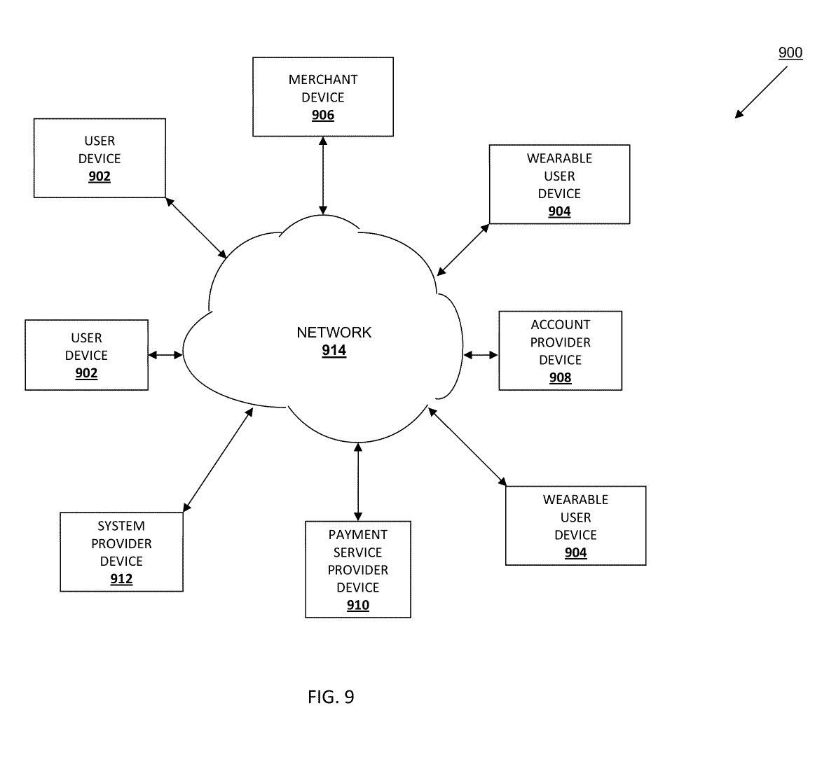

Referring now to FIG. 9, an embodiment of a network-based system 900 for implementing one or more processes described herein is illustrated. As shown, the network-based system 900 may comprise or implement a plurality of servers and/or software components that operate to perform various methodologies in accordance with the described embodiments. Exemplary servers may include, for example, stand-alone and enterprise-class servers operating a server OS such as a MICROSOFT® OS, a UNIX® OS, a LINUX® OS, or other suitable server-based OS. It can be appreciated that the servers illustrated in FIG. 9 may be deployed in other ways and that the operations performed and/or the services provided by such servers may be combined or separated for a given implementation and may be performed by a greater number or fewer number of servers. One or more servers may be operated and/or maintained by the same or different entities.

The embodiment of the networked system 900 illustrated in FIG. 9 includes a plurality of user devices 902, a plurality of wearable user devices 904, a plurality of merchant devices 906, a plurality of account provider devices 908, a payment service provider device 910, and/or a system provider device 912 in communication over one or more networks 914. The user devices 902 and wearable user devices 904 may be the user devices and/or wearable user devices discussed above and may be operated by the users discussed above. The merchant devices 906 may be the merchant devices and/or beacon devices discussed above and may be operated by the merchants discussed above. The account provider devices 908 may be the account provider devices discussed above and may be operated by the account providers discussed above. The payment service provider device 910 may be the payment service provider devices discussed above and may be operated by a payment service provider such as, for example, PayPal Inc. of San Jose, Calif. The system provider devices 912 may be the system provider devices discussed above and may be operated by the system providers discussed above.

The user devices 902, wearable user devices 904, merchant devices 906, account provider devices 908, payment service provider device 910, and/or system provider device 912 may each include one or more processors, memories, and other appropriate components for executing instructions such as program code and/or data stored on one or more computer readable mediums to implement the various applications, data, and steps described herein. For example, such instructions may be stored in one or more computer readable mediums such as memories or data storage devices internal and/or external to various components of the system 900, and/or accessible over the network 914.

The network 914 may be implemented as a single network or a combination of multiple networks. For example, in various embodiments, the network 914 may include the Internet and/or one or more intranets, landline networks, wireless networks, and/or other appropriate types of networks.

The user devices 902 may be implemented using any appropriate combination of hardware and/or software configured for wired and/or wireless communication over network 914. For example, in one embodiment, the user devices 902 may be implemented as a personal computer of a user in communication with the Internet. In other embodiments, the user devices 902 may be a smart phone, personal digital assistant (PDA), laptop computer, and/or other types of computing devices.

The user devices 902 may include one or more browser applications which may be used, for example, to provide a convenient interface to permit the user to browse information available over the network 914. For example, in one embodiment, the browser application may be implemented as a web browser configured to view information available over the Internet.

The user devices 902 may also include one or more toolbar applications which may be used, for example, to provide user-side processing for performing desired tasks in response to operations selected by the user. In one embodiment, the toolbar application may display a user interface in connection with the browser application.

The user devices 902 may further include other applications as may be desired in particular embodiments to provide desired features to the user devices 902. In particular, the other applications may include a payment application for payments assisted by a payment service provider through the payment service provider device 910. The other applications may also include security applications for implementing customer-side security features, programmatic customer applications for interfacing with appropriate application programming interfaces (APIs) over the network 914, or other types of applications. Email and/or text applications may also be included, which allow user payer to send and receive emails and/or text messages through the network 914. The user devices 902 includes one or more user and/or device identifiers which may be implemented, for example, as operating system registry entries, cookies associated with the browser application, identifiers associated with hardware of the user devices 902, or other appropriate identifiers, such as a phone number. In one embodiment, the user identifier may be used by the payment service provider device 910 to associate the user with a particular account as further described herein.

The merchant devices 906 may be maintained, for example, by a conventional or on-line merchant, conventional or digital goods seller, individual seller, and/or application developer offering various products and/or services in exchange for payment to be received conventionally or over the network 914. In this regard, the merchant devices 904 may include a database identifying available products and/or services (e.g., collectively referred to as items) which may be made available for viewing and purchase by the user.

The merchant devices 906 also include a checkout application which may be configured to facilitate the purchase by the payer of items. The checkout application may be configured to accept payment information from the customer through the user devices 902 and/or from the payment service provider through the payment service provider device 910 over the network 914.

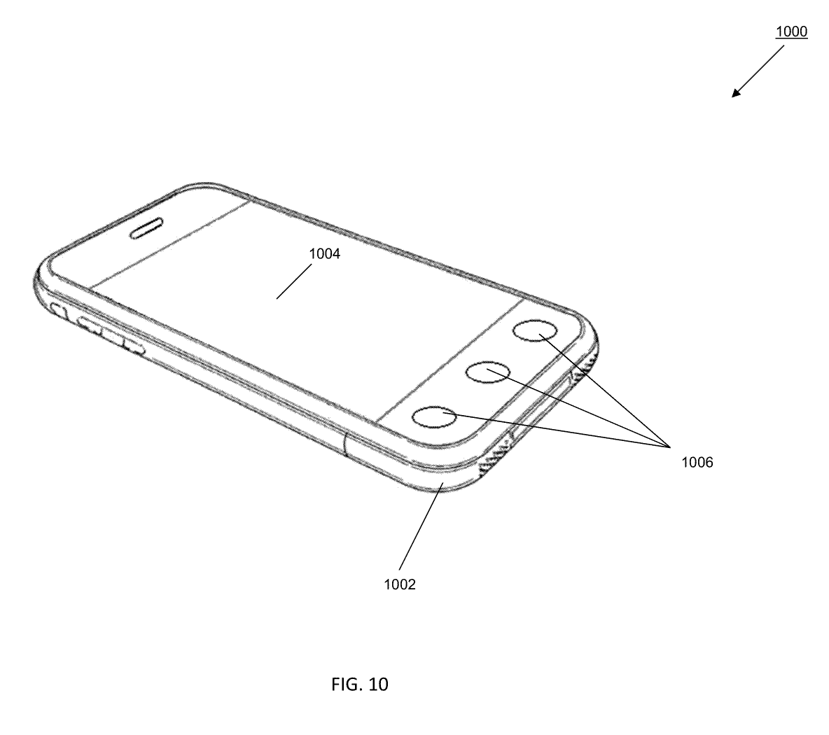

Referring now to FIG. 10, an embodiment of a user device 1000 is illustrated. The user device 1000 may be the user devices discussed above. The user device 1000 includes a chassis 1002 having a display 1004 and an input device including the display 1004 and a plurality of input buttons 1006. One of skill in the art will recognize that the user device 1000 is a portable or mobile phone including a touch screen input device and a plurality of input buttons that allow the functionality discussed above with reference to the methods above.

However, a variety of other portable/mobile user devices and/or desktop user devices may be used in the methods discussed above without departing from the scope of the present disclosure.

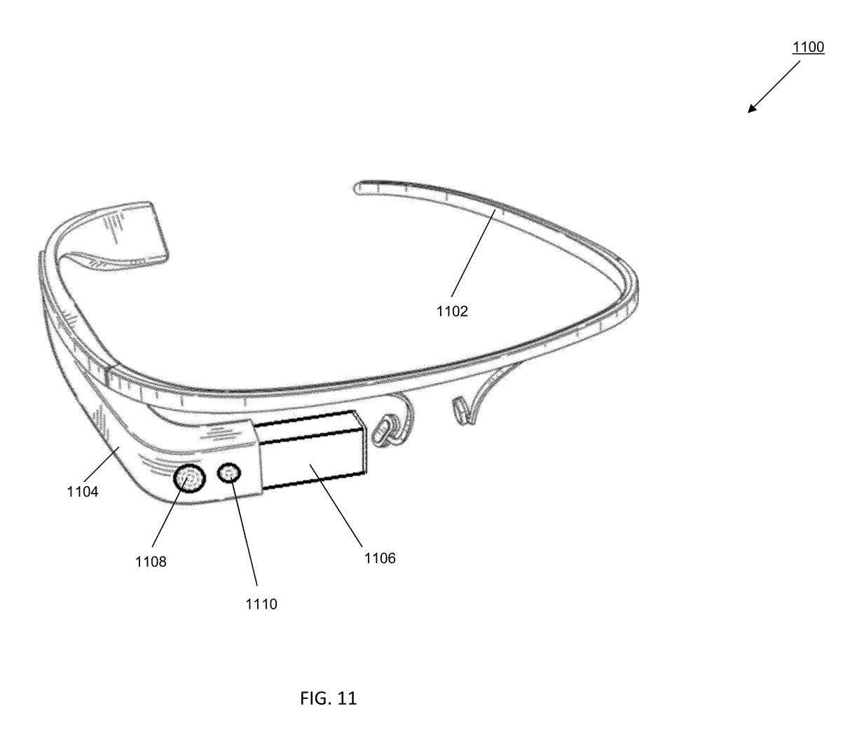

Referring now to FIG. 11, an embodiment of a wearable user device 1100 is illustrated. The wearable device 1100 may be the wearable user devices, discussed above. The wearable device 1100 includes a frame 1102 having a computing chassis 1104 that extends from the frame 1102, a display device 1106 that extends from the computing chassis 1104, a microphone 1108 located on the computing chassis 1104, and a camera 1110 located on the computing chassis 1104. One of skill in the art will recognize that the wearable merchant device 1100 is a mobile wearable merchant device such as, for example, the “smart” glasses 202 discussed above that may provide a user with the functionality discussed above with reference to the methods discussed above. However, a variety of other mobile wearable devices (e.g., smart watches, smart rings, smart shoes, etc.) may be used in the methods discussed above without departing from the scope of the present disclosure.

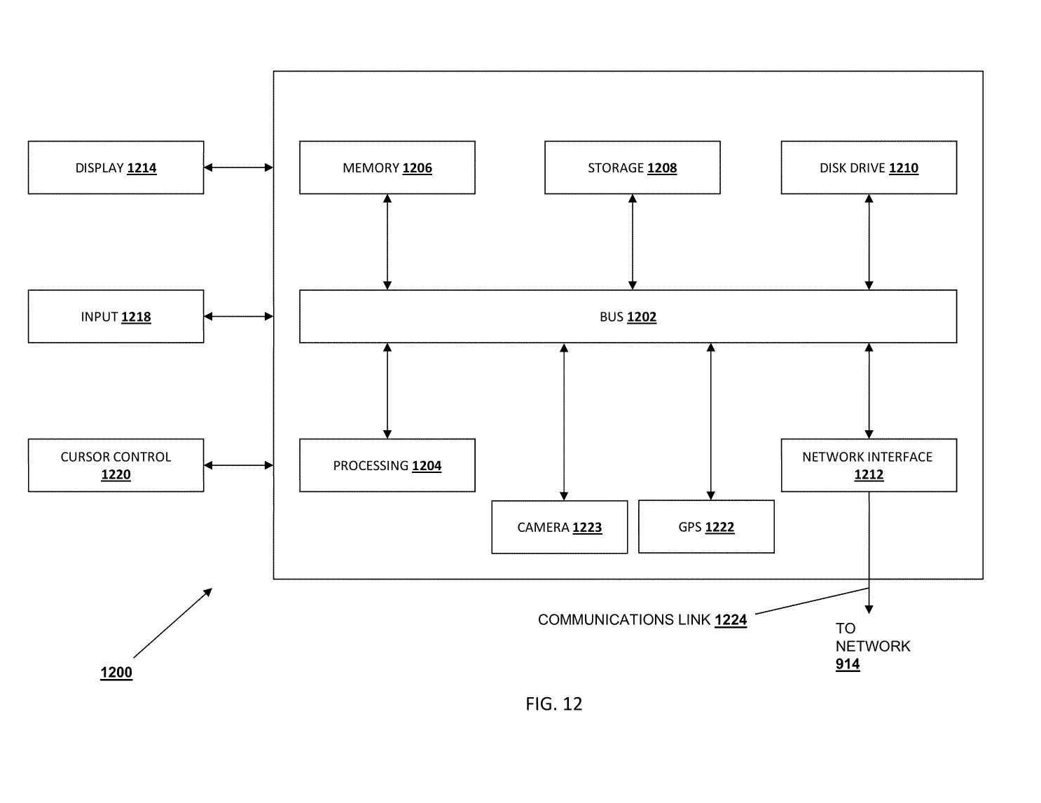

Referring now to FIG. 12, an embodiment of a computer system 1200 suitable for implementing, for example, the user devices, merchant devices, beacon devices, account provider devices, payment service provider device, and/or system provider device, is illustrated. It should be appreciated that other devices utilized by users, merchants, beacon devices, other devices, service providers, and/or system providers in the system discussed above may be implemented as the computer system 1200 in a manner as follows.

In accordance with various embodiments of the present disclosure, computer system 1200, such as a computer and/or a network server, includes a bus 1202 or other communication mechanism for communicating information, which interconnects subsystems and components, such as a processing component 1204 (e.g., processor, micro-controller, digital signal processor (DSP), etc.), a system memory component 1206 (e.g., RAM), a static storage component 1208 (e.g., ROM), a disk drive component 1210 (e.g., magnetic or optical), a network interface component 1212 (e.g., modem or Ethernet card), a display component 1214 (e.g., CRT or LCD), an input component 1218 (e.g., keyboard, keypad, or virtual keyboard), a cursor control component 1220 (e.g., mouse, pointer, or trackball), a location determination component 1222 (e.g., a Global Positioning System (GPS) device as illustrated, a cell tower triangulation device, and/or a variety of other location determination devices known in the art), and/or a camera component 1223. In one implementation, the disk drive component 1210 may comprise a database having one or more disk drive components.

In accordance with embodiments of the present disclosure, the computer system 1200 performs specific operations by the processor 1204 executing one or more sequences of instructions contained in the memory component 1206, such as described herein with respect to the user devices, merchant devices, beacon devices, other devices, payment service provider devices, and/or system provider devices. Such instructions may be read into the system memory component 1206 from another computer readable medium, such as the static storage component 1208 or the disk drive component 1210. In other embodiments, hard-wired circuitry may be used in place of or in combination with software instructions to implement the present disclosure.

Logic may be encoded in a computer readable medium, which may refer to any medium that participates in providing instructions to the processor 1204 for execution. Such a medium may take many forms, including but not limited to, non-volatile media, volatile media, and transmission media. In one embodiment, the computer readable medium is non-transitory. In various implementations, non-volatile media includes optical or magnetic disks, such as the disk drive component 1210, volatile media includes dynamic memory, such as the system memory component 1206, and transmission media includes coaxial cables, copper wire, and fiber optics, including wires that comprise the bus 1202. In one example, transmission media may take the form of acoustic or light waves, such as those generated during radio wave and infrared data communications.

Some common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, carrier wave, or any other medium from which a computer is adapted to read. In one embodiment, the computer readable media is non-transitory.

In various embodiments of the present disclosure, execution of instruction sequences to practice the present disclosure may be performed by the computer system 1200. In various other embodiments of the present disclosure, a plurality of the computer systems 1200 coupled by a communication link 1224 to the network 914 (e.g., such as a LAN, WLAN, PTSN, and/or various other wired or wireless networks, including telecommunications, mobile, and cellular phone networks) may perform instruction sequences to practice the present disclosure in coordination with one another.

The computer system 1200 may transmit and receive messages, data, information and instructions, including one or more programs (i.e., application code) through the communication link 1224 and the network interface component 1212. The network interface component 1212 may include an antenna, either separate or integrated, to enable transmission and reception via the communication link 1224. Received program code may be executed by processor 1204 as received and/or stored in disk drive component 1210 or some other non-volatile storage component for execution.

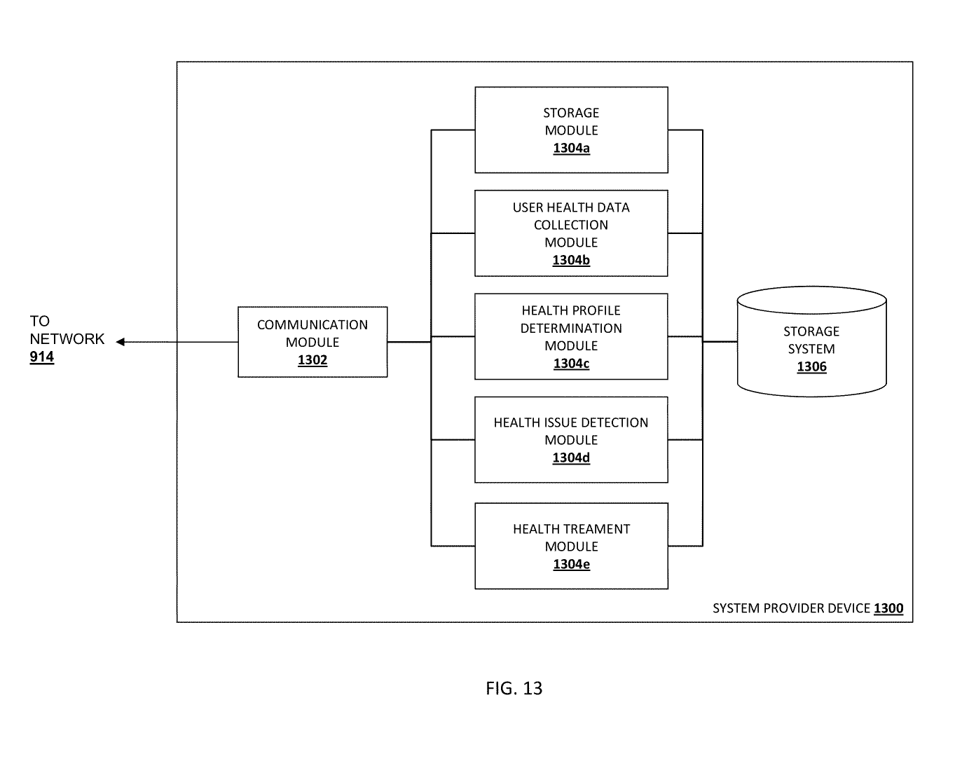

Referring now to FIG. 13, an embodiment of a system provider device 1300 is illustrated. In an embodiment, the device 1300 may be the system provider devices discussed above. The device 1300 includes a communication module 1302 that is coupled to the network 914 and to any or all of a storage module 1304a, a user health data collection module 1304b, a health profile determination module 1304c, a health issue detection module 1304d, and a health treatment module 1304e, any of which may be coupled to a storage system 1304e. Any or all of the modules 1302 and 1304a-e may be implemented as a subsystem of the system provider device including for example, a circuit, a hardware component, a hardware subcomponent, and/or a variety of other subsystems known in the art. Furthermore, any or all of the modules 1302 and 1304a-e may be preconfigured to perform their disclosed functionality, or may be configured by a processing system “on-the-fly” or as needed to perform their disclosed functionality. As such, any or all of the modules 1302 and 1304a-e may include pre-configured and dedicated circuits and/or hardware components of the system provider device 1300, or may be circuits and/or hardware components that are configured as needed.

For example, any or all of the modules 1302 and 1304a-e may be provided via one or more circuits that include resistors, inductors, capacitors, voltage sources, current sources, switches, logic gates, registers, and/or a variety of other circuit elements known in the art. One or more of the circuit elements in a circuit may be configured to provide the circuit(s) that cause the modules 1302, 1304a, 1304b, 1304c, 1304d, and/or 1304e to perform the functions described above. As such, in some embodiments, preconfigured and dedicated circuits may be implemented to perform the functions of the modules 1302, 1304a, 1304b, 1304c, 1304d, and/or 1304e. In other embodiments, a processing system may execute instructions on a non-transitory, computer-readable medium to configure one or more circuits as needed to perform the functions of the modules 1302, 1304a, 1304b, 1304c, 1304d, and/or 1304e.

The communication module 1302 may be included as a separate module provided in the device 1300, or may be provided using instructions stored on a computer-readable medium that, when executed by a processing system in the device 1300, configure the communication module 1302 to send and receive information over the network 914, as well as provide any of the other functionality that is discussed above. The storage module 1304a may be included as a separate module provided in the device 1300, or may be provided using instructions stored on a computer-readable medium that, when executed by a processing system in the device 1300, configure the storage module 1304a to store user health data and user health profiles in the storage system 1306, as well as provide any of the other functionality that is discussed above. The user health data collection module 1304b may be included as a separate module provided in the device 1300, or may be provided using instructions stored on a computer-readable medium that, when executed by a processing system in the device 1300, configure the user health data collection module 1304b to retrieve user health data at a plurality of different times, as well as provide any of the other functionality that is discussed above.

The health profile determination module 1304c may be included as a separate module provided in the device 1300, or may be provided using instructions stored on a computer-readable medium that, when executed by a processing system in the device 1300, configure the health profile determination module 1304c to analyze previous user health data retrieved at a plurality of previous times to create a user health profile, as well as provide any of the other functionality that is discussed above. The health issue detection module 1304d may be included as a separate module provided in the device 1300, or may be provided using instructions stored on a computer-readable medium that, when executed by a processing system in the device 1300, configure the health issue detection module 1304d to compare current user health data retrieved at a current time to detect a user health deviation, as well as provide any of the other functionality that is discussed above. The health treatment module 1304e may be included as a separate module provided in the device 1300, or may be provided using instructions stored on a computer-readable medium that, when executed by a processing system in the device 1300, configure the health treatment module 1304e to provide the user health treatment for display to the first user, as well as provide any of the other functionality that is discussed above. Furthermore, other modules discussed above but not illustrated in FIG. 13 may be provided as separate modules on the device 1300, or using instructions stored on a computer-readable medium similarly as discussed above. While the storage system 1306 has been illustrated as located in the device 1300, one of skill in the art will recognize that it may include multiple storage devices and may be connected to the modules 1304a-e through the network 914 without departing from the scope of the present disclosure.

Where applicable, various embodiments provided by the present disclosure may be implemented using hardware, software, or combinations of hardware and software. Also, where applicable, the various hardware components and/or software components set forth herein may be combined into composite components comprising software, hardware, and/or both without departing from the scope of the present disclosure. Where applicable, the various hardware components and/or software components set forth herein may be separated into sub-components comprising software, hardware, or both without departing from the scope of the present disclosure. In addition, where applicable, it is contemplated that software components may be implemented as hardware components and vice-versa.

Software, in accordance with the present disclosure, such as program code and/or data, may be stored on one or more computer readable mediums. It is also contemplated that software identified herein may be implemented using one or more general purpose or specific purpose computers and/or computer systems, networked and/or otherwise. Where applicable, the ordering of various steps described herein may be changed, combined into composite steps, and/or separated into sub-steps to provide features described herein.

The foregoing disclosure is not intended to limit the present disclosure to the precise forms or particular fields of use disclosed. As such, it is contemplated that various alternate embodiments and/or modifications to the present disclosure, whether explicitly described or implied herein, are possible in light of the disclosure. For example, the above embodiments have focused on merchants and users; however, a user or consumer can pay, or otherwise interact with any type of recipient, including charities and individuals. The payment does not have to involve a purchase, but may be a loan, a charitable contribution, a gift, etc. Thus, merchant as used herein can also include charities, individuals, and any other entity or person receiving a payment from a customer. Having thus described embodiments of the present disclosure, persons of ordinary skill in the art will recognize that changes may be made in form and detail without departing from the scope of the present disclosure. Thus, the present disclosure is limited only by the claims.

What is claimed is:

1. A health issue detection and treatment system, comprising:
a storage module that stores a first user health profile associated with a first user;
a user health data collection module that retrieves user health data at a plurality of different times from at least one wearable user device associated with the first user;
a health profile determination module that analyzes previous user health data retrieved by the user health data collection module from the at least one wearable user device associated with the first user at a plurality of previous times to create the first user health profile that is stored in the storage module;
a health issue detection module that compares current user health data retrieved by the user health data collection module from the at least one wearable user device associated with the first user at a current time to the first user health profile and, in response, detects a first user health deviation; and
a health treatment module that provides a first user health treatment for notification to a first user device that is associated with the first user.

2. The system of claim 1, further comprising:
a user connection module that determines a connection between the first user and a second user, wherein the health treatment module provides the first user health treatment for notification to a second user device that is associated with a second user based on the connection determined between the first user and the second user.

3. The system of claim 2, wherein the user connection module provides the first user health treatment for notification to the second user device in response to retrieving a calendar from the second user device and using the calendar to determine that the second user is available to perform the first user health treatment.

4. The system of claim 1, wherein the storage module stores a plurality of purchase suggestions in association with respective user health profiles and user health deviations, and wherein the system further comprises:
a purchase treatment module that retrieves at least one purchase suggestion from the plurality of purchase suggestions in the storage module using the first user health profile and the first user health deviation, wherein the health treatment module provides the at least one purchase suggestion as part of the first user health treatment.

5. The system of claim 1, wherein the user device is in communication with at least one user physical treatment system, and wherein the first user health treatment provides for the activation of the at least one user physical treatment system.

6. The system of claim 5, wherein the at least one user physical treatment system includes a magnetic treatment system.

7. A method for health issue detection and treatment, comprising:
collecting, by a processing system from at least one wearable user device associated with a first user, current user health data at a current time;
retrieving, by the processing system from a non-transitory memory system, a first user health profile;
comparing, by the processing system, the current user health data retrieved from the at least one wearable user device associated with the first user at the current time with the first user health profile retrieved from the non-transitory memory and, in response, detecting a first user health deviation; and
providing, by the processing system for notification to a first user device that is associated with the first user, a first user health treatment.

8. The method of claim 7, further comprising:
collecting, by the processing system from at least one wearable user device associated with a first user, previous user health data at a plurality of previous times;
analyzing, by the processing system, the previous user health data retrieved from the at least one wearable user device associated with the first user at the plurality of previous times to create the first user health profile;
storing, by the processing system in a non-transitory memory system, the first user health profile.

9. The method of claim 8, wherein the user health data collecting by the processing system from the at least one wearable user device associated with a first user includes physiological user data.

10. The method of claim 7, further comprising:
receiving, by the processing system, a plurality of second user purchases associated with a plurality of second users that each include a second user health profile and a second user health deviation; and
storing, by the processing system in the non-transitory memory system, the plurality of second user purchases in association with respective second user health profiles and second user health deviations.

11. The method of claim 10, further comprising:
retrieving, by the processing system from the non-transitory memory system, at least one of the plurality of second user purchases based the first user health profile and the first user health deviation matching at least one second user health profile and second user health deviation; and
providing at least one purchase suggestion using the at least one of the plurality of second user purchases, wherein the at least one purchase suggestion is provided as part of the first user health treatment.

12. The method of claim 7, wherein the processing system is in communication with at least one user physical treatment system, and wherein the first user health treatment provides for the activation of the at least one user physical treatment system.

13. The method of claim 12, wherein the at least one user physical treatment system includes an electric current treatment system.

14. A non-transitory computer-readable medium comprising instructions which, in response to execution by a computer system, cause the computer system to:
configure a health data collection circuit to retrieve user health data at a plurality of different times from at least on wearable user device associated with a first user;
configure a profile determination circuit to analyze previous user health data retrieved by the health data collection circuit from the at least one wearable user device associated with the first user at a plurality of previous times to create a first user health profile;
configure a storage circuit to store the first user health profile in a database;
configure a detection circuit to compare current user health data retrieved by the health data collection circuit from the at least one wearable user device associated with the first user at a current time and, in response, detect a first user health deviation; and
configure a treatment circuit to provide a first user health treatment for notification to a first user device that is associated with the first user.

15. The non-transitory machine-readable medium of claim 14 further comprising instructions which, in response to execution by a computer system, cause the computer system to:
configure a user connection circuit that determines a connection between the first user and a second user, retrieves a calendar of the second user, and determines that the second user is currently available, and wherein the treatment circuit provides the first user health treatment for notification to a second user device that is associated with a second user in response to determining the connection determined between the first user and the second user and that the second user is currently available.

16. The non-transitory machine-readable medium of claim 14, wherein the user health data collected by the processing system from the at least one wearable user device associated with a first user includes user movement data.

17. The non-transitory machine-readable medium of claim 14, wherein the storage circuit stores a plurality of purchase suggestions that have been previously determined to have an effect on user health deviations that are substantially similar to the first user health deviation, and wherein the non-transitory machine-readable medium further comprising instructions which, in response to execution by a computer system, cause the computer system to:
configure a purchase treatment circuit that retrieves at least one purchase suggestion from the plurality of purchase suggestions in the database using the first user health deviation, wherein the treatment circuit provides the at least one purchase suggestion as part of the first user health treatment.

18. The non-transitory machine-readable medium of claim 14, further comprising instructions which, in response to execution by a computer system, cause the computer system to:
configure a user physical treatment circuit, wherein the first user health treatment provides for the activation of the user physical treatment circuit.

19. The non-transitory machine-readable medium of claim 18, wherein the user physical treatment circuit includes a user support device physical adjustment circuit.

20. The non-transitory machine-readable medium of claim 19, wherein the user support device physical adjustment circuit is included in a pair of shoes worn by the first user.
